(19) United States
(12) Patent Application Publication
(10) Pub. No.: US 2023/0263425 A1
(43) Pub. Date: Aug. 24, 2023

(54) TEMPORAL SAMPLING IN A WEARABLE BREATH ANALYSER

(71) Applicant: Calibre Biometrics Inc., Wellesley Hills, MA (US)

(72) Inventor: Udi E. Meirav, Waban, MA (US)

(73) Assignee: Calibre Biometrics Inc., Wellesley Hills, MA (US)

(21) Appl. No.: 18/310,102

(22) Filed: May 1, 2023

Related U.S. Application Data
(63) Continuation of application No. PCT/US2021/055999, filed on Oct. 21, 2021.
(60) Provisional application No. 63/108,570, filed on Nov. 2, 2020.

(51) Int. Cl. A61B 5/08; A61B 5/00; A61B 5/097; A61B 5/087

(52) U.S. Cl. CPC A61B 5/082; A61B 5/6803; A61B 5/097; A61B 5/087

(57) ABSTRACT

Systems and methods are disclosed for temporal sampling of air exhaled by a user. Some embodiments comprise a mask adapted to be worn by a user, a chamber, disposed in or on the mask, the chamber comprising a valve, at least one sensor, disposed in the chamber, for measuring composition of air, an actuator adapted to open the valve, and at least one processor. The at least one processor is programmed to selectively instruct the actuator to open the valve, thereby causing air in the mask to be exposed to the at least one sensor, at a time associated with exhalation by the user, and to selectively instruct the actuator to close the valve, thereby preventing air from entering the chamber, at a time associated with inhalation by the user.

### RELATED APPLICATIONS

This application is a continuation of commonly assigned International Patent Application No. PCT/US2021/055999, entitled TEMPORAL SAMPLING IN A WEARABLE BREATH ANALYSER, filed Oct. 21, 2021, bearing Attorney Docket No. V0340.70002WO00, which claims the benefit under 35 U.S.C. § 119(e) of the filing date of U.S. Provisional patent application Ser. No. 63/108,570, entitled TEMPORAL SAMPLING IN A WEARABLE BREATH ANALYSER, filed Nov. 2, 2020, bearing Attorney Docket No. V0340.70002US00. The entirety of each of the documents listed above is incorporated herein by reference.

### TECHNICAL FIELD

Embodiments of the present disclosure generally relate to systems, methods and devices for collecting, analyzing and utilizing respiratory, physiological, metabolic or biometric data.

### BACKGROUND

Respiratory air composition can be a useful metric for many applications including medical, physical conditioning and nutrition. Typically, this is done by collecting exhaled air from the subject directly exhaling into a collection tube, or wearing a breathing mask attached to a tube with a directional valve that physically separates exhaled air from inhaled air; the exhaled air is conveyed to an analyzer configured with sensors that can measure concentration of one or more components of the air, such as oxygen or carbon dioxide. The use of tubes for physical separation of exhaled air from ambient air generally leads to a more cumbersome and intrusive system that not only makes these measurements more difficult, but ultimately deters subjects and health professionals from more widespread use of such breath measurements.

### BRIEF DESCRIPTION OF THE DRAWINGS

Various aspects and embodiments of the invention are described herein with reference to the following figures. It should be appreciated that the figures are not necessarily drawn to scale. Items appearing in multiple figures are indicated by the same reference number in all the figures in which they appear. In the figures:

FIG. 1 is a schematic diagram of a representative wearable breath analyser in which some embodiments of the invention may be implemented;

FIGS. 2-5 each schematically depict a representative analyzer subsystem, in accordance with some embodiments of the invention;

FIG. 6 is an expanded view of a representative actuator, in accordance with some embodiments of the invention;

FIG. 7 is a side view of a representative actuator, in accordance with some embodiments of the invention;

FIGS. 8-9 are side views of a representative flap in a closed and an open position, respectively, in accordance with some embodiments of the invention;

FIGS. 10-11 are depictions in which a flap rests in a closed position, and is forced open by an actuator, respectively, in accordance with some embodiments of the invention;

FIG. 12 is a flowchart depicting a representative method for controlling temporal sampling, in accordance with some embodiments of the invention;

FIG. 13 is a flowchart depicting a representative process for sampling a subject's breath over a plurality of breathing cycles, in accordance with some embodiments of the invention; and

FIG. 14 is a graph showing CO₂ concentration by sampling delay, in accordance with some embodiments of the invention; and

FIG. 15 is a block diagram depicting a representative computing system comprising components that may be used to implement embodiments of the invention.

### DETAILED DESCRIPTION

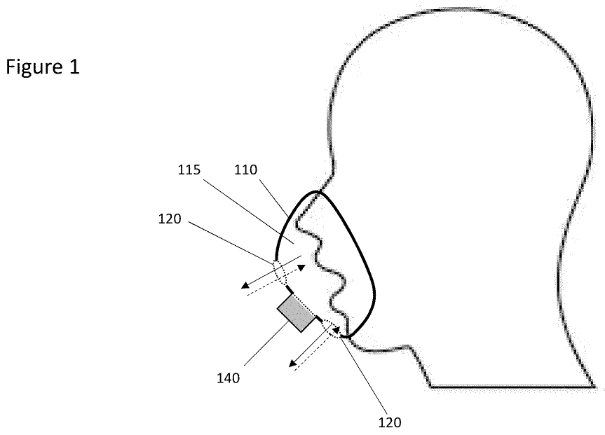

A schematic diagram of a wearable analyser in which some embodiments of the invention may be implemented is shown in FIG. 1. The schematic figure shows a breathing mask (110) worn on a subject's face. The mask allows inhalation and exhalation through one or more apertures or inlets (120) in the mask. A mask may comprise any suitable quantity of apertures. The aperture(s) open directly to the ambient air and allow air to flow in and out, during inhalation and exhalation, respectively. In some embodiments of the invention, a breathing aperture may be attached to a tube or conduit or any other air flow element (not shown). There can be any number of such inlets or conduits on a single mask, but generally there is no mechanism separating inhalation or exhalation so as to flow through different pathways. A sensing or analyzing subsystem (140) is further attached to the system, and as will be readily understood, it is not generally required for it to be located so as to intercept the respiratory air flow along the air flow path. The arrows in FIG. 1 depict schematically the flow of inhaled (dashed line) and exhaled (solid line) air.

According to one aspect of the invention, the composition of exhaled air may be measured without having a spatially different flow paths for inhaled and exhaled air with sensors located only in the path of exhaled air (where separate exhaled air properties would then be measured). In certain embodiments of the invention, the exhaled air and the inhaled air essentially flow through the same “shared” pathways. However, the system exploits the fact that inhalation and exhalation cannot occur at the same time, and therefore exhaled air composition can be determined by measuring the composition of the air stream only during specific times which correspond to exhalation. In other words, the outgoing exhaled air is allowed essentially to flow through the same path as the incoming inhaled air between the ambient space and the respiratory openings (namely, the mouth and the nose); however, some embodiments of the invention are configured to selectively allow exhaled air to be exposed to the sensing and analyzing subsystem at a time corresponding to exhalation by the user, thereby allowing for measurement of the composition of air during certain time intervals that coincide with exhalation, and specifically with the flow of exhaled in the immediate vicinity of the analyzer. As a result, some embodiments of the invention enable selective measurement of only exhaled air, in a mask which need not include separate passageways for air to be inhaled by the user and air exhaled by the user. As will become clear, in some embodiments the measurement interval need not be the entire exhalation time and may comprise only a shorter interval that is only part of the exhalation time.

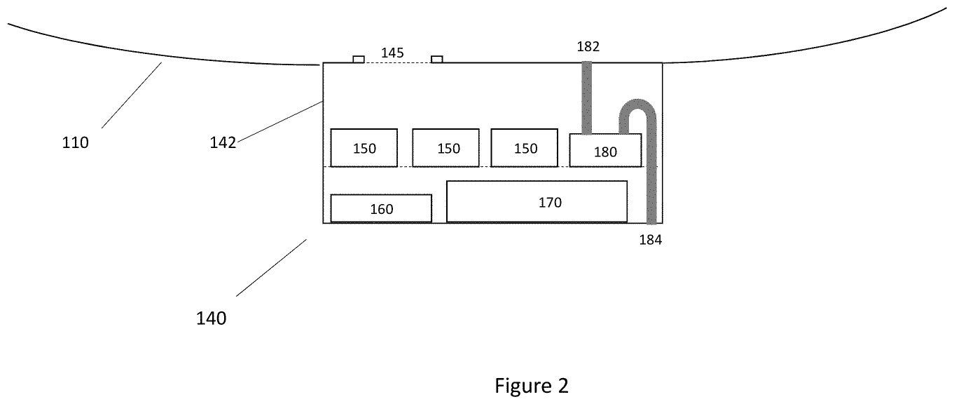

An analyzer subsystem (140) for use in some embodiments is shown in an expanded schematic in FIG. 2. It comprises a housing (142) with an air permeable aperture (145), and one or more gas sensors (150) that are supported by a microprocessor (160) and a battery (170) or other power source. The gas sensors can be configured to detect oxygen, nitrogen, carbon dioxide (CO₂), humidity, temperature, or any aerosol, vapors or trace components of the air. Exhaled air is known to contain hundreds of types of molecules, also knowns in the art as bio-effluents. In some embodiments the microprocessor and the power source can be physically part of the subsystem, while in other embodiments one or more of these elements can be located elsewhere with an electrical or wireless (radio) connection to the sensors. Although only three gas sensors 150, one microprocessor 160 and one battery 170 are shown, an analyzer subsystem may comprise any suitable quantity of these components.

In some embodiments a pressure sensor or a directional or bi-directional flow sensor is used to detect the direction of air flow F(t), namely exhalation (F>0) versus inhalation (F<0). The embodiment of FIG. 2 shows a two-port differential pressure sensor (180) configured to measure the pressure difference between a point inside the mask (182) and a point outside the mask (184). This is just one example of any number of possible configuration measuring pressure difference between two points along the air flow path. During exhalation, the higher pressure point is “upstream” relative to the exhalation flow direction, thus the microprocessor can determine at any point in time whether the subject is inhaling or exhaling. In other embodiments other types of flow sensors can be used, as long as a sensor provides the microprocessor the time-dependent flow information F(t) with which the gas sensor (150) readings are harmonized. In some embodiments the flow value F may not need to be quantitatively accurate, as long as it has the correct sign (+/−) to distinguish between exhalation and inhalation.

The harmonization of sampling by the gas sensors with the breath cycle, and specifically with the exhalation time, can be done digitally, mechanically or electromechanically, as will be explained. In this respect, the term “harmonization” may refer to the sampling being at least partially synchronized with exhalation, occurring during a period of time which corresponds in some way with the start of exhalation, occurring during a period of time which corresponds in some way with the rate of exhalation air flow, or bears any other suitable temporal relationship(s) with one or more events occurring during the exhalation process. The choice of whether to harmonize sampling with exhalation digitally, mechanically or electromechanically may in part be dependent on the response time of the specific sensors (150) and the measurement requirements.

Digital harmonization may be performed, for example, if a sensor has a response time which is fast relative to the typical duration of the breathing cycles. In this case the microprocessor can receive sensor readings continuously and, using the flow sensor readings F separately store or analyze the gas sensor readings that coincide with positive (negative) values of F and are therefore associated with exhaled (inhaled) air, respectively, and thus allow subsequent computation or display corresponding separately and specifically to exhaled (inhaled) air, respectively.

However, some gas sensors have response times which may not allow for digitally harmonized measurement of gas properties with the respiratory cycle. There are several factors that may influence a particular sensor's response time. In some cases, the diffusion time can cause slow response in many types of gas sensors, and often leads to response times that are longer than the typical human breath cycle. In other cases, electronic response time or digital integration can contribute to sensor delays. Many CO₂ non-dispersive infrared (NDIR) sensors have response times as high as 30 seconds or more. Similarly, electrochemical sensors used for oxygen and various trace gases have response times measured in many seconds, not milliseconds. In these embodiments, a digital harmonization approach may not provide adequate temporal selectivity between inhaled and exhaled breath; electromechanically assisted harmonization can provide a better solution, as explained ahead. Of course, the invention is not limited in this respect, and the selection of digital, electromechanical, and/or other types of components for use in measuring air composition may be influenced by any of numerous factors, which may include but are not limited to sensor response time.

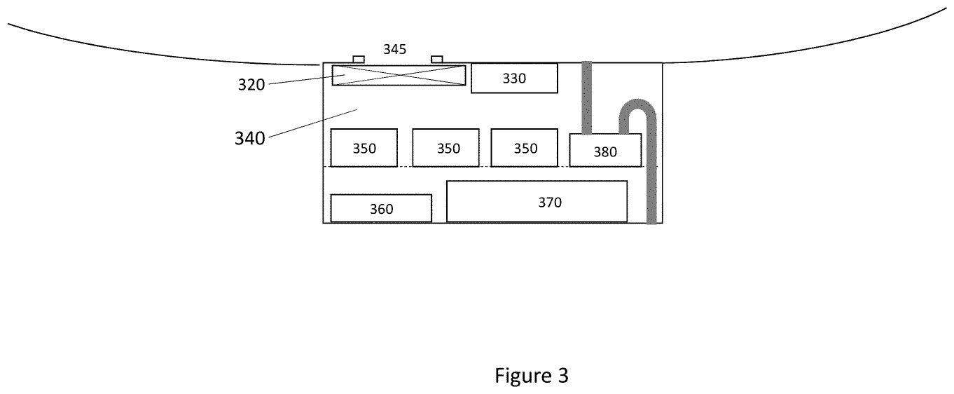

FIG. 3 illustrates schematically an embodiment of electromechanical harmonization. Here the gas sensors are configured to sense the air composition in a small enclosure or “sensing chamber” (340) that is in fluid communications with the respiratory air flow through a small aperture (345) with a valve or shutter (320). In some embodiments the valve is controlled directly or indirectly, through an actuator (330) controlled by the microprocessor (360) and designed to open only during certain time intervals. A simple example would be the time intervals associated with the flow of exhaled air, as determined by the flow sensor (380) and signaled to the microprocessor. The valve actuator can rely on any suitable mechanism for converting an electrical signal to mechanical motion, including but not limited to piezoelectric force, an electromagnetic force, and electrostatic force, an electric motor, or a MEMS (micro-electromechanical system) chip.

In some embodiments, when the subject exhales, a positive pressure (dP>0) is generated inside the breathing mask by the subject's exhalation and sensed by the differential pressure sensor (380). The microprocessor (360) receives the positive pressure reading and sends an instruction to the valve actuator (330) to open the valve (320) to the sensing chamber (340). The interior of the sensing chamber (340) is still not part of the primary flow path of the exhaled air, so the opening of the valve allows some of the exhaled air to enter into the chamber and come into direct contact with the sensors (350). The mixing of exhaled air into the chamber can be caused by a combination of contributing mechanisms including but not limited to diffusion, turbulence, pressure gradients, and the movement of the valve itself. When the exhalation ends, the pressure reading is reversed, and the microprocessor signals the valve to shut. Thus over time the chamber's air content contains exclusively exhaled air. So while the air composition in the respiratory flow path changes as the subject repeatedly inhales and exhales, the sensors are exposed almost exclusively to recently-exhaled air. The sensors continually take readings and thereby update the recorded composition parameters they are designed to sense, and if physiological processes lead the composition of exhaled air to change over time, the sensors will continuously track these changes, without having the values mingled with those of inhaled air composition, the latter being essentially just ambient air.

In certain embodiments, the valve and actuator combination responsible for controlling the coupling between the main respiratory flow path and the gas sensors may be selected so as to meet certain desired characteristics, such as compact size, low power consumption, low cost, reliability, and/or fast response time. That said, any suitable valve(s) and/or actuator(s) may be employed.

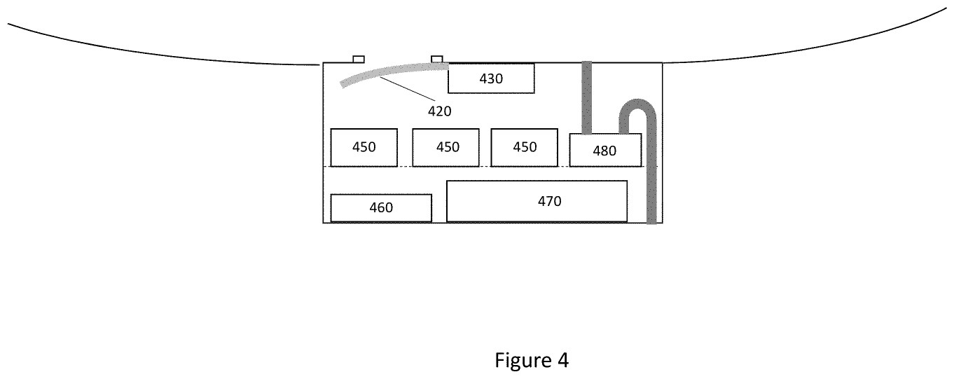

In some embodiments, the aperture may be sealed with the help of a piezoelectric actuator where a voltage applied to an electrode on the piezoelectric element causes piezoelectric forces that move a seal that can cover the aperture. An example is shown in FIG. 4. The seal (420) comprises a piece of piezoelectric material that is normally flat and configured to cover the aperture, possibly with some additional sealing element, resulting in an air-tight cover of the aperture. When the material is energized by a voltage from the actuator (430), the seal bends and lifts away from the aperture, thereby opening it to air flow. Other embodiments of the piezoelectrically-actuated seal are possible and can be designed to meet the geometrical form of the aperture and the space available for the actuator and the seal.

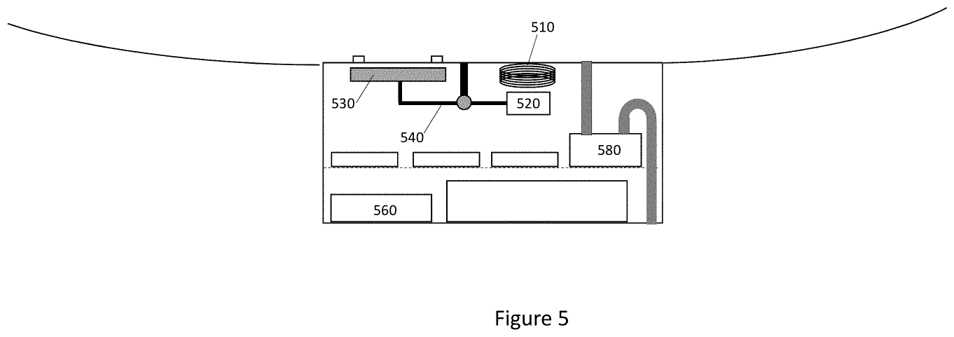

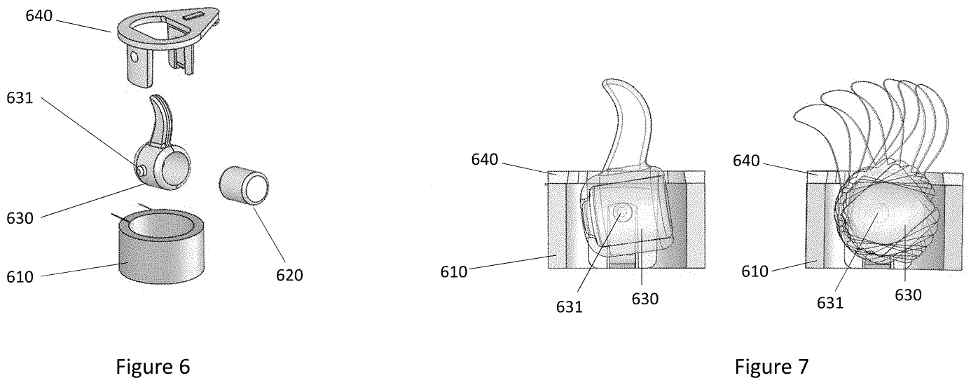

In one embodiment, shown schematically in FIG. 5 and in more detail in FIGS. 6-11, the actuator utilizes an electromagnet coil (510) controlled by the microprocessor, so that the electromagnet can apply a magnetostatic force on a fixed ferromagnetic or paramagnetic element (520), which in turn is attached to a valve (530) that can seal the aperture.

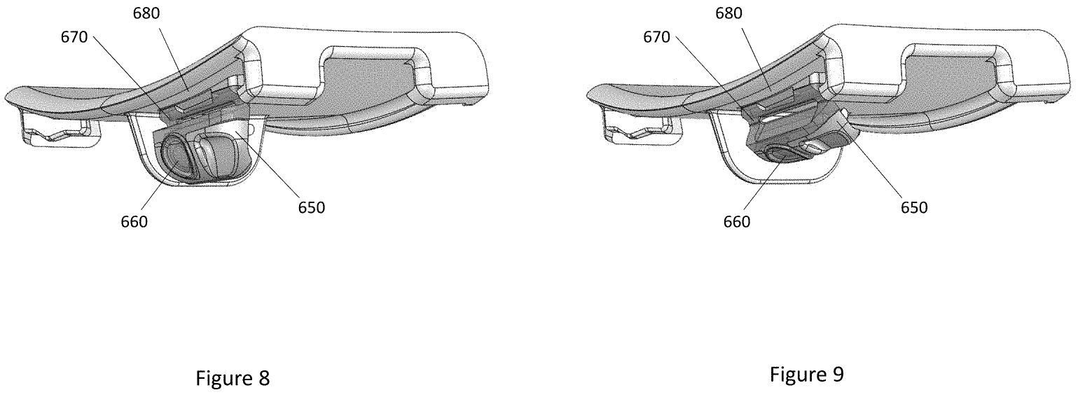

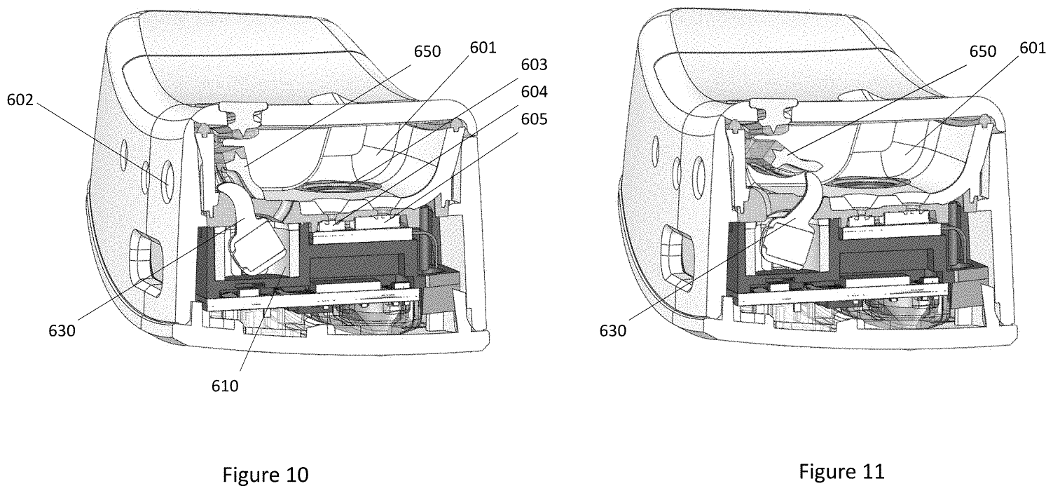

FIGS. 6-11 shows in more detail a representative embodiment employing the magnetic actuation mechanism shown schematically in FIG. 5. The actuator comprises several elements viewed in FIG. 6, including an electromagnetic coil (610) and a fixed ferromagnetic cylindrical element (620) that is embedded in a lever arm (630) that is attached through two hinged joints (631) to a supporting frame (640). As seen in FIG. 7, the coil (610) is fixed to the frame (640) while the lever (630) can rotate on the hinges. When a current is generated in the coil, a magnetostatic force is induced between the coil and the embedded ferromagnet, which moves the latter while rotating the lever on the hinges. A representative seal is shown in FIGS. 8-9. It comprises a flap (650) with a sealing surface (660) and elastic attachment (670) to a fixed base (680). The elastic material can be rubber, plastic, metal, or any other suitable material and/or form. The material may be configured so that the elastic forces draw the flap towards the chamber inlet, so that its sealing surface naturally tends to mate with the aperture and seals the aperture, as shown in FIG. 8. As will be seen more clearly in FIG. 10-11, the lever may not be attached to the flap, but may be capable of moving it. The elastic attachment (670) is designed to bend, allowing the flap to be pushed away from the aperture as shown in FIG. 9 when the lever arm pushes it in that direction. FIGS. 10-11 show a cross section of a representative system in this embodiment, including the sensing chamber (601) and the inlet aperture (602) and gas sensors (603), (604) and (605) in fluid communication with the sensing chamber. When the electromagnetic coil (610) is off, the lever arm (630) applies no force and the natural elasticity of the flap (650) brings it towards the inlet aperture and seals it, as shown in FIG. 6e. When the actuator coil (610) is energized, as shown in FIG. 6f, the lever (630) rotates and pushes the sealing flap (650) away from the aperture and thus allowing air to flow through the aperture into the sensing chamber (601). The microprocessor can therefore open and close the aperture with an electric signal that controls current flow through the coil.

In some embodiments, including the one shown in FIGS. 6-11, the elastic element provides a tensile or elastic force that naturally closes the valve when no magnetic force is present, while the magnetic force—when actuated—counteracts the tensile force and opens the valve. The reverse configuration is also possible, where the tensile force is designed to keep the valve open in the absence of the counteracting magnetic force and only seal the aperture when the magnetic force is applied. In some embodiments the seal material provides the elasticity required without a separate spring mechanism.

In some embodiments elasticity does not play a significant role in opening or closing the valve, and both directions or motion are controlled by magnetostatic force. This can be achieved in any number of suitable ways. In some embodiments the actuating force is reversed by reversing the polarity of the electromagnet which can be done by changing the direction of current flowing through the coil. In some embodiments a fixed magnet provides a constant force in one direction, and when the opposite force is required, an electromagnet provides a larger, reverse force that overrides the fixed magnet.

Other mechanisms can be used to open and close the aperture shutter, including but not limited to electrostatic force, an electric motor, and a MEMS (micro-electromechanical system) actuator. As an example of an electrostatic actuator, a pair of electrodes forming a capacitor is configured with one fixed and the other attached to a moving element that can directly or indirectly cause the valve to move from an open to a closed position. Thus a voltage that is controlled by the microprocessor and applied between the electrostatic actuator electrodes can cause the valve to open and close as needed.

In a separate example, a miniature servo motor or a stepper motor controls the valve position. Servo motors weighing as little as 1 gram and only a few millimeters in any dimension are commercially available and can be incorporated into the sensing subsystem on a wearable breathing mask, while being controlled by the onboard microprocessor. While more complex and intricate than a simple electrostatic or magnetostatic force, they offer other advantages such as better controlled speed and predetermined range of motion.

### Timing the Sampling Of Exhaled Air

The timing at which the actuator opens the valve need not coincide exactly with the initial detection of exhalation. In reference to a specific inhalation cycle labeled by the index n, the time where exhalation begins (as detected by the pressure or flow sensor) is denoted as t=Xₙ, and the time when the microprocessor signals the actuator to open as t=X′ₙ. In some embodiments the opening of the valve is signaled immediately upon detection of an exhalation signal from the pressure or flow sensor. In this scenario X′ₙ=Xₙ. Commercially available differential pressure sensors have response times as short as a few milliseconds (ms) or less, which is virtually instantaneous relative to the characteristic times of respiratory cycles.

In other embodiments, the microprocessor is programmed for a certain time interval, or delay, dₙ between X′ₙ and Xₙ, namely X′ₙ=Xₙ+dₙ. There could be any number of reasons for such intentional delay, including but not limited to (a) providing time for a sufficient amount of exhaled air to flow so as to flush out and displace incoming air, from a previous inhalation, in the vicinity of the aperture, (b) sampling air at a later stage of each exhalation, which is predominantly alveolar air from deeper in the lungs rather than from the respiratory “dead space”, and (c) generally shortening the amount of time the valve is open to reduce excess ingress and condensation of water near the sensors.

Similarly the valve need not remain open until the end of exhalation, but only for a duration of time D that may be shorter than the time remaining until the end of exhalation. Typically exhalation flow rate F(t), sometime called the expiratory flow rate (EFR), rises at the beginning of each exhalation cycle (n), reaches a peak value F′ₙ, and then gradually declines until reaching zero, and eventually reverses as the next inhalation begins. The microprocessor can be programmed to close that valve at a time X″ₙ=X′ₙ+Dₙ (before the end of exhalation), based on any number of considerations or algorithms, and in particular based on the value of F(t) or a derivative of the flow rate, such as dF/dt. As a few non-limiting examples, the valve can be programmed to seal at one of, or the sooner of, the following (a) Once the exhalation flow rate begins to decline and/or reaches a value that is less than a certain percentage of F′ₙ (i.e., its peak value in that cycle); or (b) after a fixed duration D of being open.

In other embodiments the opening delay (d) or the duration (D) can further depend on the specific measured values of F′ₙ, which is typically higher under rapid breathing. For example, when F′ₙ is higher, Dₙ may be shorter.

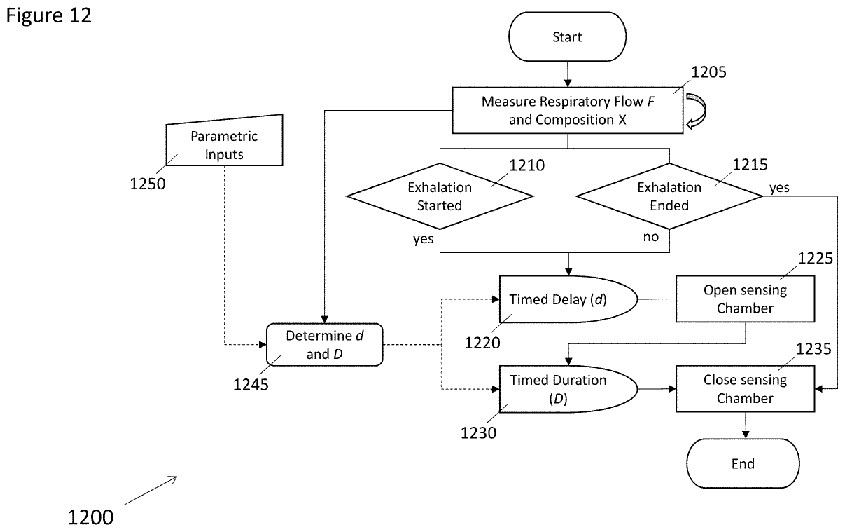

FIG. 12 shows a flow chart depicting a representative process 1200 for controlling the temporal sampling window in accordance with some embodiments of the invention. At the start of representative process 1200, respiratory flow F and composition X are continually monitored at 1205 (e.g., as described above). Upon a determination (e.g., based on F) at 1210 that exhalation has started, a sampling occurrence begins at 1225 by opening a sensing chamber after a certain delay d (indicated at 1220) relative to the detection of exhalation. Sampling then continues until the earlier of (i) a calculated duration D (indicated at 1230) or (ii) the end of that particular exhalation cycle (indicated at 1215), whereupon the sensing chamber is closed (indicated at 1235). As indicated at 1245, the values of d and D and may be fixed or may change with each breath, and may be determined (e.g., calculated) based upon any combination of external inputs and measured breath quantities (indicated at 1250), including but not limited to flow, pressure, composition or frequency (breathing rate). After the sensing chamber is closed, representative process 1200 ends.

Notwithstanding the description of certain embodiments provided above, it should be appreciated that the sampling and measurement of exhaled air at particular times need not be accomplished by forcing open the valve at those times. For example, some embodiments may enable sampling of exhaled air at particular times by forcing the valve closed at other times, and/or by using other techniques. Any of numerous techniques may be used to enable sampling of exhaled air at particular times, and the invention is not limited to any particular mode of implementation.

The ability to dynamically control the timing of the sampling, both in terms of its start after a delay (d) relative to the onset of the exhalation cycle as well as its duration (D), can be used for improved measurement or for further insight into the respiratory metrics and pulmonary health of the subject. In some embodiments, these (e.g., software-based) controls, or “settings”, can be changed dynamically, even within the course of the same measurement session, to provide different information on the on subject's respiration and health. In one embodiment the microprocessor may be programmed, or instructed by another computing device, to dynamically change d and D over a progression of values and generate or analyze readings on the subject with different values of these settings.

There can be several reasons or benefits in changing the values of d or D during a measurement period which spans a certain time period or a sequence of multiple breaths. In certain embodiments, the duration D is adjusted based on the expiratory flow rate (EFR), to allow for the desired amount of air to reach the sensors. When the subject is breathing more vigorously, less exposure time may be required for the same amount of air to flow to the sensors, and thus D can be shorter, with the benefit such as limiting the amount of airborne humidity that is carried into the sensing chamber and could condense into liquid droplets near the sensors. In the same example, when the subject is breathing more slowly, a longer exposure time may be required for a sufficient amount of exhaled air to displace the same amount of air in the vicinity of the sensors.

As a non-limiting example of an adjustable D, the oxygen and/or CO₂ sensor is configured to measure the air in a small sub chamber attached to the mask that received exhaled air from the user through a controlled aperture with a calibrated or calculated pressure-vs-flow characteristic, such that at a pressure P the flow into the chamber is F_{c}(P). Furthermore the chamber volume, V_{c}, is known and constant. Thus the time it takes to displace the volume of his chamber is approximately V_{c}/F_{c} and so the value of D can be determined dynamically as D≅V_{c}/F_{c} or more generally D=a×V_{c}/F_{c} where a is a multiplicative dimensionless coefficient that can be set by the system programmer.

In another analogous, non-limiting example, the delay d can also be adjusted based on the total breath flow rate F or the cumulative flow from the beginning of the exhalation cycle. In this example the reason could be to ensure that the air filling the “dead volume” of the mask at the end of the last inhalation (and possibly including the air that has entered the bronchi but not the lungs) is fully displaced by exhaled air from within the lungs before air is sampled by the sensors. The dead volume V can be associated with the volume confined between the mask and the user's face, which is shown schematically in FIG. 1, where it is labeled (115). Alternatively the dead volume can be defined more broadly to further include the anatomical dead volume, namely the volume of air in the breathing passages leading to the lungs, such as the oral or nasal cavities as well as the bronchial tubes. The time lag required to displace the dead volume can be approximated as the ratio d=V/F, or more generally d=a×V/F where a is a multiplicative coefficient that need not be exactly 1.

The mathematical similarity between the dependence of D on F and that of d on F is evident, but to recap and for avoidance of confusion, D may be motivated by the displacement of the air from the previous exhalation in the sensing chamber, whereas d may be motivated by the displacement of the air in the “dead space” that was drawn in the preceding inhalation.

The following examples use the nomenclature (d,D) to characterize each setting, with these quantities expressed in seconds. In other words, (0.5, 1.2) implies that for each breath, the air sampling valve opens 0.5 seconds after positive differential pressure, i.e. exhalation, is first detected, and closes 1.2 seconds later.

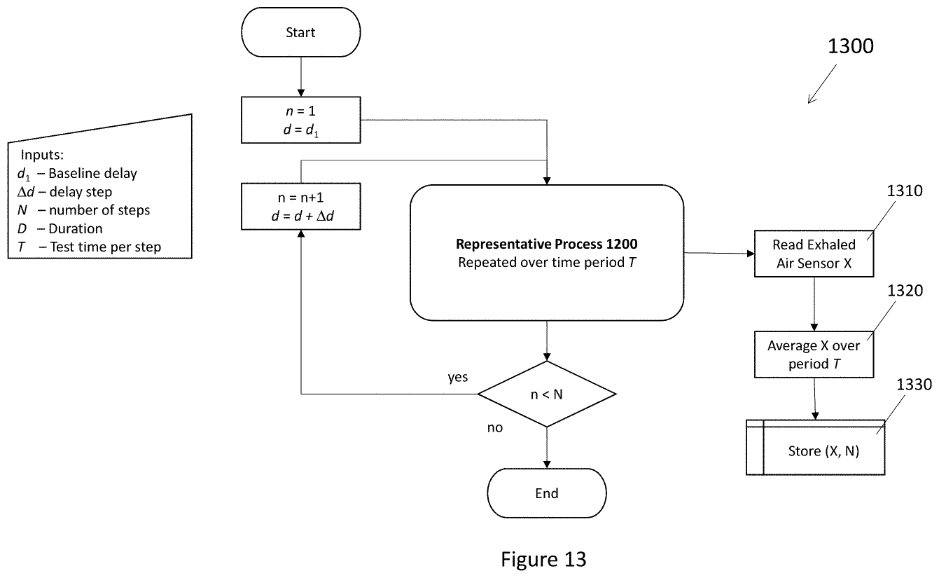

In some embodiments, a human subject's respiration may be tested continuously over a period of time corresponding to many breath cycles, so that the delay d is repeated for a number of breaths and then increased or otherwise modified. FIG. 13 shows a flowchart depicting a representative process 1300 for performing repeated sampling occurrences over a measurement period in accordance with these embodiments. In process 1300, multiple samples N are collected over a time period T, by repeating the representative process 1200 described above with reference to FIG. 12. At each sampling occurrence, a sampling delay d may be varied (e.g., increased). At each sample, one or more properties X of the exhaled breath composition (e.g., CO₂ concentration) may be read (as indicated at 1310), averaged over T (as indicated at 1320) and stored in association with the sample (as indicated at 1330).

As an illustrative non-limiting example, the system may conduct a series of consecutive measurements, each lasting 3 minutes, with the settings as follows:

TABLE 1

| Clock (mm:ss) | | Sampling Delay d | Sampling Duration D |
|---|---|---|---|
| Start | End | (sec) | (sec) |
| 00:00 | 01:00 | 0.25 | 0.25 |
| 01:00 | 02:00 | 0.50 | 0.25 |
| 02:00 | 03:00 | 0.75 | 0.25 |
| 03:00 | 04:00 | 1.00 | 0.25 |
| 04:00 | 05:00 | 1.25 | 0.25 |
| 05:00 | 06:00 | 1.50 | 0.25 |
| 06:00 | 07:00 | 1.75 | 0.25 |
| 07:00 | 08:00 | 2.00 | 0.25 |
| 08:00 | 09:00 | 2.25 | 0.25 |
| 09:00 | 10:00 | 2.50 | 0.25 |

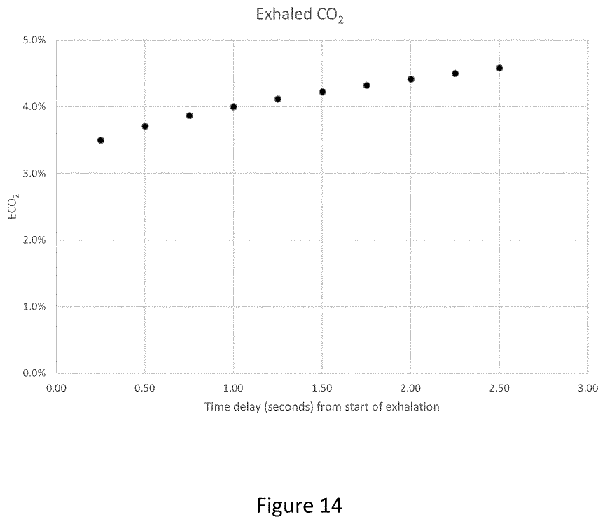

FIG. 14 shows CO₂ concentration vs sampling delay d corresponding to the table above. In this example each point on the chart is an aggregated average value from more than 10 consecutive breaths in a one-minute interval, as shown in the Table 1. The averaging may help improve the fidelity of the readings and eliminate noise and fluctuations, while the controlled and consistent values of d, combined with relatively short durations D, may provide better temporal resolution in the reading to time-dependent concentration of exhaled gases.

Delayed measurements may reveal different information about gas exchange in the respiratory systems. The overall values and the relative values of these different measurements can be used as a biometric and diagnostic tool for pulmonary function and health. Higher value of the initial delay, d, is expected to result in higher concentration of CO₂, known in the art as end tidal CO₂ (also known in the art as ETCO₂), and lower concentration of oxygen (similarly, end tidal oxygen). ETCO₂ is an important diagnostic in emergency medicine and for a variety of pulmonary and cardiac conditions.

Selective temporal sampling can also be used to improve detectability and diagnostic power of trace compounds in breath, some of which may be more concentrated in late stages of each exhalation and therefore easier to detect. Examples of such trace compounds include metabolites like ammonia, acetone, methanol, isoprene, ethanol and other volatile organic compounds (VOCs). These are typically found at levels well below 1 part per million (ppm). While such concentrations are readily detectable with sophisticated laboratory instruments, they may require longer sensor exposure times can be challenging to measure accurately with small, portable, battery-powered sensors, and hence a concentration boost facilitated by temporal sampling can serve to improve detectability and accuracy.

Another reason for temporal sampling with progressive sequence of delays (d) is to help distinguish between organic bio-effluents originating within the lungs—also known as endogenic VOCs, and bio-effluents originating in the mouth or nasal cavities, namely exogenic VOCs. This is a result of the fact that the earlier air is exhaled in a respiratory cycle, the more likely it is that the air originated in the physiological dead space, whereas the later air is exhaled in a respiratory cycle, the more likely it is that the air originates from within the lungs. In other words, early-tidal VOCs are more likely to be exogenic, whereas end-tidal VOCs are more likely endogenic. In fact the dependency a particular VOC concentration on timing parameters can be prima facie diagnostic evidence of the predominant source of that VOC. In one embodiment of the invention, a progressive scan of d is performed while detecting a particular VOC (e.g. ethanol or acetone), displayed on a chart analogous to FIG. 12, and the slope of the curve is compared to a benchmark and used to generate an indication whether the VOC is endogenic.

The timing of the sampling can be driven by numerous factors other than the respiratory cycle itself. In some embodiments, the sampling of exhaled air composition is associated with a stimulus which can be physical, neurological or even mental, and this can be done for purposes of research or monitoring/diagnostics. In one nonlimiting example, exhaled CO₂ concentration is related to hyperventilation and anxiety, which suggests that some embodiments may be used to measure the anxiety induced by certain stimuli, by timing the valve opening in correlation to the timing of the potentially anxiety-provoking influence. Other influences and other biomarkers (e.g., particular respiratory bio-effluents) can be correlated, studied and monitored for any desired purpose, including but not limited to research, diagnostic procedures and therapies. The influences can be physical such as medical/therapeutic procedures or drugs—whether delivered orally, intravenously, subcutaneously or any other way—as well as experiential, mental, psychological or any other type of influence that may cause a direct or indirect respiratory indication.

It should be appreciated from the foregoing that aspects of the invention may be embodied in any of numerous forms. For example, some embodiments of the invention are directed to a system comprising: a mask adapted to be worn by a user; a chamber, disposed in or on the mask, the chamber comprising a valve; at least one sensor, disposed in the chamber, for measuring composition of air; an actuator adapted to open the valve; and at least one processor programmed to: selectively instruct the actuator to open the valve, thereby causing air in the mask to be exposed to the at least one sensor, at a time associated with exhalation by the user; and selectively instruct the actuator to close the valve, thereby preventing air from entering the chamber, at a time associated with inhalation by the user.

Some embodiments are directed to a method for use in a system comprising a mask adapted to be worn by a user, the mask comprising a chamber having a valve, at least one sensor, disposed in the chamber, for measuring composition of air, an actuator adapted to open and close the valve, and at least one processor. The method comprises acts of: (A) the at least one processor selectively instructing the actuator to open the valve, thereby causing air in the mask to be exposed to the at least one sensor, at a time associated with exhalation by the user; and (B) the at least one processor selectively instructing the actuator to close the valve, thereby preventing air from entering the chamber, at a time associated with inhalation by the user.

Other embodiments are directed to a method for use in a system comprising a mask adapted to be worn by a user, the mask comprising at least one sensor for measuring composition of air, the mask being devoid of separate passageways for air to be inhaled by the user and air exhaled by the user. The method comprises an act of: causing only air exhaled by the user to be exposed to the at least one sensor.

Other embodiments are directed to a method for use in a system comprising a mask adapted to be worn by a user, the mask comprising at least one sensor for measuring composition of air. The method comprises an act of: selectively causing air in the mask to be exposed to the at least one sensor, at a time which corresponds to exhalation by the user.

Still other embodiments are directed to a method for use in a system comprising a mask adapted to be worn by a user, the mask comprising at least one sensor for measuring composition of air. The method comprises an act of: causing an opening to the chamber to close, thereby preventing air from entering the chamber, at a time which corresponds to inhalation by the user.

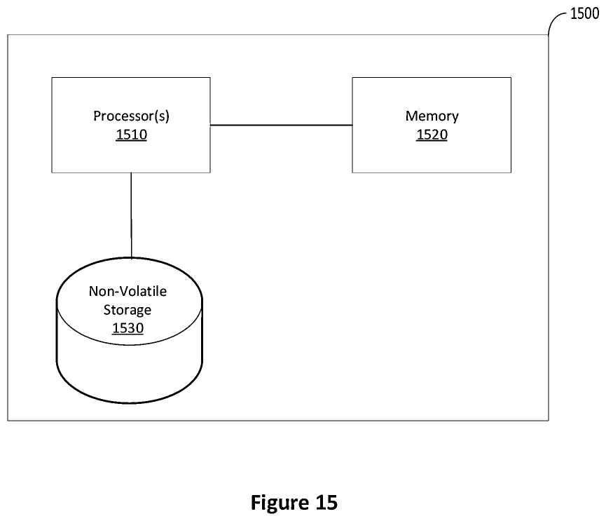

It should be appreciated from the foregoing that some embodiments may be implemented, wholly or in part, via a computing system, or one or more components thereof. A representative computing system 1500 which may be employed by various embodiments of the invention is shown in FIG. 15. The representative computing system 1500 shown includes one or more processors 1510 and one or more articles of manufacture which comprise non-transitory computer-readable storage media (e.g., memory 1520 and one or more non-volatile storage media 1530). The processor(s) 1510 may control writing data to and reading data from the memory 1520, and writing data to and reading data from the non-volatile storage device 1530, in any suitable manner, as the aspects of the disclosure provided herein are not limited in this respect. To control temporal sampling of exhaled breath, and/or perform any other functionality described herein, the processor(s) 1510 may execute one or more processor-executable instructions, which may be stored in memory 1520 and/or non-volatile storage 1530.

Example embodiments of the methods and components of the current subject matter have been described herein. These example embodiments have been described for illustrative purposes only and are not limiting. Other embodiments are possible and are covered by the current subject matter. Such embodiments will be apparent to persons skilled in the relevant art(s) based on the teachings contained herein. Thus, the breadth and scope of the current subject matter should not be limited by any of the above-described exemplary embodiments, but rather should be defined only in accordance with the following claims and their equivalents.

What is claimed is:

1. A system comprising:
a mask adapted to be worn by a user;
a chamber, disposed in or on the mask, the chamber comprising a valve;
at least one sensor, disposed in the chamber, for measuring composition of air;
an actuator adapted to open the valve; and
at least one processor programmed to:
selectively instruct the actuator to open the valve, thereby causing air in the mask to be exposed to the at least one sensor, at a time associated with exhalation by the user; and
selectively instruct the actuator to close the valve, thereby preventing air from entering the chamber, at a time associated with inhalation by the user.

2. The system of claim 1, wherein the at least one processor is programmed to selectively instruct the actuator to open the valve at a time which at least partially coincides with exhalation by the user.

3. The system of claim 2, wherein the at least one processor is programmed to selectively instruct the actuator to open the valve at a time which at least partially coincides with a start of exhalation by the user.

4. The system of claim 1, wherein the at least one processor is programmed to determine a difference between air pressure in the mask and air pressure outside the mask, and to determine based on the pressure difference that exhalation by the user is occurring.

5. The system of claim 1, wherein the at least one processor is programmed to determine a first time at which exhalation by the user begins, and to selectively instruct the actuator to open the valve a predetermined interval after the first time.

6. The system of claim 5, wherein the at least one processor is programmed to select the predetermined interval so that the air in the mask that is caused to be exposed to the at least one sensor does not originate at a dead volume.

7. The system of claim 1, wherein:
the mask is adapted to be worn by a user having a repetitive breathing cycle, each breathing
cycle comprising inhalation and exhalation by the user;
the at least one processor is programmed to selectively instruct the actuator to open the valve at times associated with exhalation during a plurality of breathing cycles; and
the interval varies for each of the plurality of breathing cycles.

8. The system of claim 7, wherein the at least one processor is programmed to:
increase the interval for each of the plurality of breathing cycles; and
determine one or more particular properties of breath exhaled by the user in each of the plurality of breathing cycles.

9. The system of claim 8, wherein the one or more particular properties that the at least one processor is programmed to determine comprises CO₂ concentration in the breath exhaled by the user in each breathing cycle.

10. The system of claim 1, wherein the at least one processor is programmed to selectively instruct the actuator to close the valve after a duration of time associated with exhalation by the user has passed since the valve was opened.

11. The system of claim 10, wherein the at least one processor is programmed to determine a rate of air flow from inside of the mask to outside of the mask as a result of exhalation by the user, and to determine the duration of time based on the rate of air flow, so that the valve is closed prior to the end of exhalation by the user.

12. The system of claim 11, wherein:
the mask is adapted to be worn by a user having a repetitive breathing cycle, each comprising inhalation and exhalation by the user;
the at least one processor is programmed to selectively instruct the actuator to open the valve at times associated with exhalation during a plurality of breathing cycles; and
the duration of time is the same for each of the plurality of breathing cycles.

13. The system of claim 1, wherein the mask is devoid of separate passageways for air to be inhaled by the user and air exhaled by the user.

14. The system of claim 1, wherein the actuator comprises:
at least one electromagnetic element;
at least one hinge; and
a lever adapted to rotate about the at least one hinge;
wherein the electromagnetic element is adapted to supply a force to the lever, causing the lever to rotate about the at least one hinge and open the valve.

15. The system of claim 1, wherein the valve comprises an element adapted to supply an elastic force tending to close the valve when not opened by the actuator.

16. A method of measuring breath composition by a user, the method being for use in a system comprising a chamber, the chamber having a valve and at least one sensor for measuring composition of air, the system comprising at least one processor and an actuator adapted to open and close the valve, the method comprising acts of:
(A) the at least one processor selectively instructing the actuator to open the valve, thereby causing respiratory air to be exposed to the at least one sensor, at a time associated with exhalation by the user; and
(B) the at least one processor selectively instructing the actuator to close the valve, thereby preventing air from entering the chamber, at a time associated with inhalation by the user.

17. The method of claim 16, wherein the act (A) comprises the at least one processor selectively instructing the actuator to open the valve at a time which at least partially coincides with exhalation by the user.

18. The method of claim 17, wherein the act (A) comprises the at least one processor selectively instructing the actuator to open the valve at a time associated with a start of exhalation by the user.

19. The method of claim 16, comprising acts, performed prior to the act (A), comprising:
determining an air pressure at one or more locations along the path of respiratory air flow; and
determining based on the pressure that exhalation by the user is occurring.

20. The method of claim 16, wherein the act (A) comprises:
determining a first time at which exhalation by the user begins; and
the at least one processor selectively instructing the actuator to open the valve a predetermined interval after the first time.

21. The method of claim 20, wherein the act (A) comprises:
determining a rate of air flow as a result of exhalation by the user; and
determining the interval based at least in part on the rate of air flow.

22. The method of claim 20, wherein the act (A) comprises selecting the predetermined interval at least in part so that the respiratory air that is selectively caused to be exposed to the at least one sensor does not originate at a dead volume.

23. The method of claim 16, wherein:
the user has a repetitive breathing cycle, each breathing cycle comprising inhalation and exhalation by the user;
the act (A) is performed for a plurality of breathing cycles; and
the interval varies for each of the plurality of breathing cycles.

24. The method of claim 23, wherein:
the interval increases for each of the plurality of breathing cycles; and
the act (A) comprises detecting one or more particular properties of breath exhaled by the user in each of the plurality of breathing cycles.

25. The method of claim 24, wherein the one or more particular properties detected in the act (A) comprises CO2 concentration in the breath exhaled by the user in each breathing cycle.

26. The method of claim 16, wherein the act (B) comprises the at least one processor selectively instructing the actuator to close the valve after a duration of time associated with exhalation by the user has passed since the valve was opened.

27. The method of claim 26, wherein the act (B) comprises:
determining a rate of air flow as a result of exhalation by the user; and
determining the duration of time based on the rate of air flow, so that the duration of time terminates prior to the end of exhalation by the user.

28. The method of claim 26, wherein:
the user has a repetitive breathing cycle, each comprising inhalation and exhalation by the user;
the acts (A) and (B) are performed for a plurality of breathing cycles; and
the duration of time varies for each of the plurality of breathing cycles.

29. The method of claim 16, wherein the system is devoid of separate passageways for air to be inhaled by the user and air exhaled by the user.
